(19) United States
(12) Patent Application Publication
(10) Pub. No.: US 2007/0299550 A1
(43) Pub. Date: Dec. 27, 2007

(54) Three-Dimensional Guidance System And Method , And Drug Delivery System

(75) Inventors: Shigehiro Nishijima, Osaka (JP); Shinichi Takeda, Osaka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: OSAKA UNIVERSITY, Suita-shi, (JP)

(21) Appl. No.: 11/575,992

(22) PCT Filed: Aug. 1, 2005
(86) PCT No.: PCT/JP05/14480

§ 371 Date: Mar. 26, 2007

(30) Foreign Application Priority Data
Sep. 28, 2004 (JP) 2004-280780

(51) Int. Cl. G05B 19/18; A61B 5/05; H01H 47/00

(52) U.S. Cl. 700/61; 361/152; 600/424

(57) ABSTRACT

A three-dimensional guidance system of the present invention includes a bed (1) horizontally driven by a bed drive motor (11), a position detecting sensor (6) for detecting a position of a magnetic particle carrier (8), a plurality of electromagnets (3, 4, 5) arranged to surround the bed (1), and a controller (7) for controlling a current to be supplied to the plurality of electromagnets (3, 4, 5) and a drive signal to be supplied to the bed drive motor (11). The controller (7) holds a vascular route as three-dimensional route data, and feedback-controls the current to be supplied to the plurality of electromagnets (3, 4, 5) and the drive signal to be supplied to the bed drive motor (11), based on a deviation of the current position of the magnetic particle carrier (8) detected by the position detecting sensor (6) from a target position.

### TECHNICAL FIELD

The present invention relates to a system and method for guiding a magnetic particle carrier along a channel extending in a certain route in three-dimensional space, and to a system for delivering a drug through a blood vessel close to an affected part.

### BACKGROUND ART

Gene therapy has been attracting attention in recent years, where has been proposed a therapeutic approach of utilizing a gene delivery factor such as a liposome to deliver a gene to a target cell part (JP 7-241192, A).

There also has been proposed, as a therapeutic approach against various diseases, means of sending a microcapsule enclosing a therapeutic drug with a catheter from a portal vein into a cancerous organ or the like to directly administer the therapeutic drug to the organ (JP 2002-516587, A).

However, the therapy of utilizing a gene delivery factor such as a liposome to deliver a gene has a drawback in that genes could be delivered also to a non-target cell. The therapy of sending a therapeutic drug using a catheter has problems not only in that the catheter insertion brings pain and danger to patients, but also in that the therapy cannot be applied to thin blood vessels into which it is difficult to insert a catheter.

Accordingly, an object of the present invention is to provide a system and method capable of guiding an object such as a therapeutic drug to a target position along a channel such as a blood vessel, without using a tool such as a catheter, and a drug delivery system.

### DISCLOSURE OF THE INVENTION

A three-dimensional guidance system of the present invention is for guiding a magnetic particle carrier along a channel extending in a certain route in three-dimensional space, and includes a magnetic field forming device for forming a magnetic field in the space having the channel therein, and a controller for controlling the magnetic field forming device operation, wherein the three-dimensional guidance system guides the magnetic particle carrier along the channel by controlling the magnetic field intensity and gradient of the magnetic field formed by the magnetic field forming means.

In the above three-dimensional guidance system of the present invention, the magnetic particle carrier in the channel will experience a magnetic force (driving force), and move in the direction of that force, depending on the intensity and gradient of the magnetic field formed by the magnetic field forming means. Thus, the magnetic particle carrier can be smoothly moved along the channel if the controller controls the magnetic field intensity and gradient so as to generate a magnetic force along the channel for the magnetic particle carrier in the channel.

Alternatively, a three-dimensional guidance system of the present invention includes a position detecting sensor for detecting the magnetic particle carrier position in the channel, a plurality of electromagnets arranged to surround the channel, a driver for displacing the plurality of electromagnets relative to the channel in a direction penetrating a plane having the electromagnets arranged therein, and a control circuit for controlling a current to be supplied to the plurality of electromagnets and a drive signal to be supplied to the driver.

The control circuit includes:

data holding means for holding the channel route as three-dimensional route data; and

feedback control means for feedback-controlling the current to be supplied to the plurality of electromagnets and the drive signal to be supplied to the driver, based on a deviation of position data representing the current position of the magnetic particle carrier detected by the position detecting sensor from the route data held by the data holding means.

In the above three-dimensional guidance system of the present invention, the plurality of electromagnets generate lines of magnetic force, which form a magnetic field having a plurality of magnetic fields superposed in the space including the channel, so that the magnetic particle carrier experiences a magnetic force with a magnitude and direction depending on the intensity and gradient of the magnetic field. Consequently, the magnetic particle carrier will be driven and moved by the magnetic force.

During this process, the feedback control adjusts the current to be supplied to the plurality of electromagnets and the drive signal to be supplied to the driver so as to bring the deviation of the magnetic particle carrier position data from the route data (target value), i.e., the position difference of the magnetic particle carrier relative to a predetermined route along the channel, close to zero. Therefore, the magnetic particle carrier moves in the channel along the predetermined route regardless of the action of gravity or other external forces on the magnetic particle carrier. Even if the magnetic particle carrier momentarily becomes unstable in position, the magnetic particle carrier will quickly be stable again and move along the predetermined route because of the feedback control.

Although Earnshaw's theorem states that a magnet cannot stand still stably under a static magnetic field, it is possible to move a magnetic particle carrier along a predetermined route by employing the feedback control in moving the magnetic particle carrier depending on the intensity and gradient of the magnetic field, as in the above present invention.

Alternatively, a three-dimensional guidance system of the present invention is for guiding a magnetic particle carrier, injected into a blood vessel in a body, along the blood vessel, and includes a magnetic field forming device for forming a magnetic field in space having the body therein, and a controller for controlling the magnetic field forming device operation, wherein the three-dimensional guidance system guides the magnetic particle carrier along the blood vessel by controlling the magnetic field intensity and gradient of the magnetic field formed by the magnetic field forming device.

In the above three-dimensional guidance system of the present invention, the magnetic particle carrier is injected into the blood vessel by an injector. Thereafter, the magnetic particle carrier in the blood vessel will experience a magnetic force (driving force), and move in the direction of that force, depending on the intensity and gradient of the magnetic field formed by the magnetic field forming means. Thus, the magnetic particle carrier can be smoothly moved along the blood vessel if the controller controls the magnetic field intensity and gradient so as to generate a magnetic force along the blood vessel for the magnetic particle carrier in the blood vessel.

Alternatively, a three-dimensional guidance system of the present invention includes a position detecting sensor for detecting the magnetic particle carrier position in the blood vessel, a plurality of electromagnets to be arranged to surround the body, a driver for displacing the plurality of electromagnets relative to the body in a direction penetrating a plane having the plurality of electromagnets arranged therein, and a control circuit for controlling a current to be supplied to the plurality of electromagnets and a drive signal to be supplied to the driver.

The control circuit includes:

data holding means for holding the blood vessel route extending in the body as three-dimensional route data; and

feedback control means for feedback-controlling the current to be supplied to the plurality of electromagnets and the drive signal to be supplied to the driver, based on a deviation of position data representing the current position of the magnetic particle carrier detected by the position detecting sensor from the route data held by the data holding means.

In the above three-dimensional guidance system of the present invention, the plurality of electromagnets generate lines of magnetic force, which form a magnetic field having a plurality of magnetic fields superposed in the space including the body, so that the magnetic particle carrier experiences a magnetic force with a magnitude and direction depending on the intensity and gradient of the magnetic field. Consequently, the magnetic particle carrier will be driven and moved by the magnetic force.

During this process, the feedback control adjusts the current to be supplied to the plurality of electromagnets and the drive signal to be supplied to the driver so as to bring the deviation of the magnetic particle carrier position data from the route data (target value), i.e., the position difference of the magnetic particle carrier relative to the blood vessel route, close to zero. Therefore, the magnetic particle carrier moves in the blood vessel along the predetermined route regardless of the action of gravity or other external forces on the magnetic particle carrier. Even if the magnetic particle carrier momentarily becomes unstable in position, the magnetic particle carrier will quickly be stable again and move along the predetermined route because of the feedback control. Then, finally, the magnetic particle carrier reaches a target organ or cell part.

Specifically, the driver is for moving a bed one-dimensionally by driving a bed drive motor, and the plurality of electromagnets are arranged to surround the bed, in a plane perpendicular to the bed moving direction. In this specific configuration, the magnetic particle carrier is position-controlled in one dimension by control of the bed drive motor, and position-controlled in two dimensions perpendicular to the one dimension by control of the magnetic force of the plurality of electromagnets.

Specifically, the feedback control means of the control circuit prepares a current signal depending on the current to be supplied to the plurality of electromagnets, and a voltage signal depending on the drive signal to be supplied to the driver, based on the deviation, and supplies the current signal through a current amplifier to each of the electromagnets, and the voltage signal to the bed drive motor.

Further specifically, the magnetic particle carrier includes a drug or biological molecule carrying a magnetic particle, and further specifically, includes a microcapsule enclosing a drug or biological molecule with a magnetic particle. The magnetic particle contains one or more metals selected from iron, nickel and cobalt, or compounds of these metals. With this specific configuration, after the magnetic particle carrier reaches a target organ or cell part, the drug or biological molecule will flow out of the microcapsule, and be administered to the organ or cell part with a high local concentration. The microcapsule itself is gradually absorbed in the body. The magnetic particle is gradually decomposed and metabolized in the body.

A drug delivery system of the present invention is for delivering drug particles injected into a blood vessel in a body along the blood vessel close to an affected part, each drug particle including a drug or biological molecule carrying a magnetic particle, the drug delivery system including a magnetic field forming device for forming a magnetic field in space having the body therein, and a controller for controlling the magnetic field forming device operation, wherein the system controls the magnetic field intensity and gradient of the magnetic field formed by the magnetic field forming device, and thereby guides the drug particles along a predetermined vascular route close to the affected part, where the particles accumulate and aggregate. The magnetic field forming device includes a super-conducting magnet, for example. A driver is also provided for varying the magnetic field forming device position relative to the body.

For example, at a vascular furcation from one main vessel to a plurality of branch vessels, a magnetic field gradient where the magnetic field increases in strength from the inside of the blood vessel toward the outside is formed near one branch vessel into which the drug particles are to be sent, whereby the drug particles concentratedly flow into the one branch vessel. This allows the drug particles injected into a vein by an injector or the like to selectively pass through one of the branches of a vascular system including veins and arteries, and to be delivered to or close to the affected part along the predetermined vascular route. A magnetic field gradient where the magnetic field increases in strength from the inside of the blood vessel toward the outside is then formed near the affected part, whereby the drug particles in the blood vessel accumulate and aggregate at or near the affected part. This allows the drugs to be administered to the affected part with a high local concentration.

As described above, the present invention can smoothly guide an object such as a therapeutic drug to a target position along a channel such as a blood vessel without using a toot such as a catheter.

### BRIEF DESCRIPTION OF THE DRAWINGS

FIG. 1 is a perspective view showing a configuration of a three-dimensional guidance system of the present invention.

FIG. 2 is a front view showing arrangements and structures of three electromagnets.

FIG. 3 illustrates a configuration of a magnetic particle carrier.

FIG. 4 is a control block diagram of a three-dimensional guidance system of the present invention.

FIG. 5 is a sectional view illustrating drug particles selectively flowing into one branch vessel at a vascular bifurcation.

FIG. 6 is a sectional view illustrating drug particles accumulating at a certain intravascular position.

FIG. 7 is a graph showing relationships between the diameter of drug particles and the magnetic field gradient, necessary for the drug particles to accumulate at a certain intravascular position.

FIG. 8 is a diagram illustrating a magnetic field position that allows drug particles to selectively flow into one branch vessel at a vascular bifurcation.

### BEST MODE FOR CARRYING OUT THE INVENTION

The present invention embodied in a three-dimensional guidance system as a therapeutic unit will be specifically described below with reference to the drawings.

Three-Dimensional Guidance System

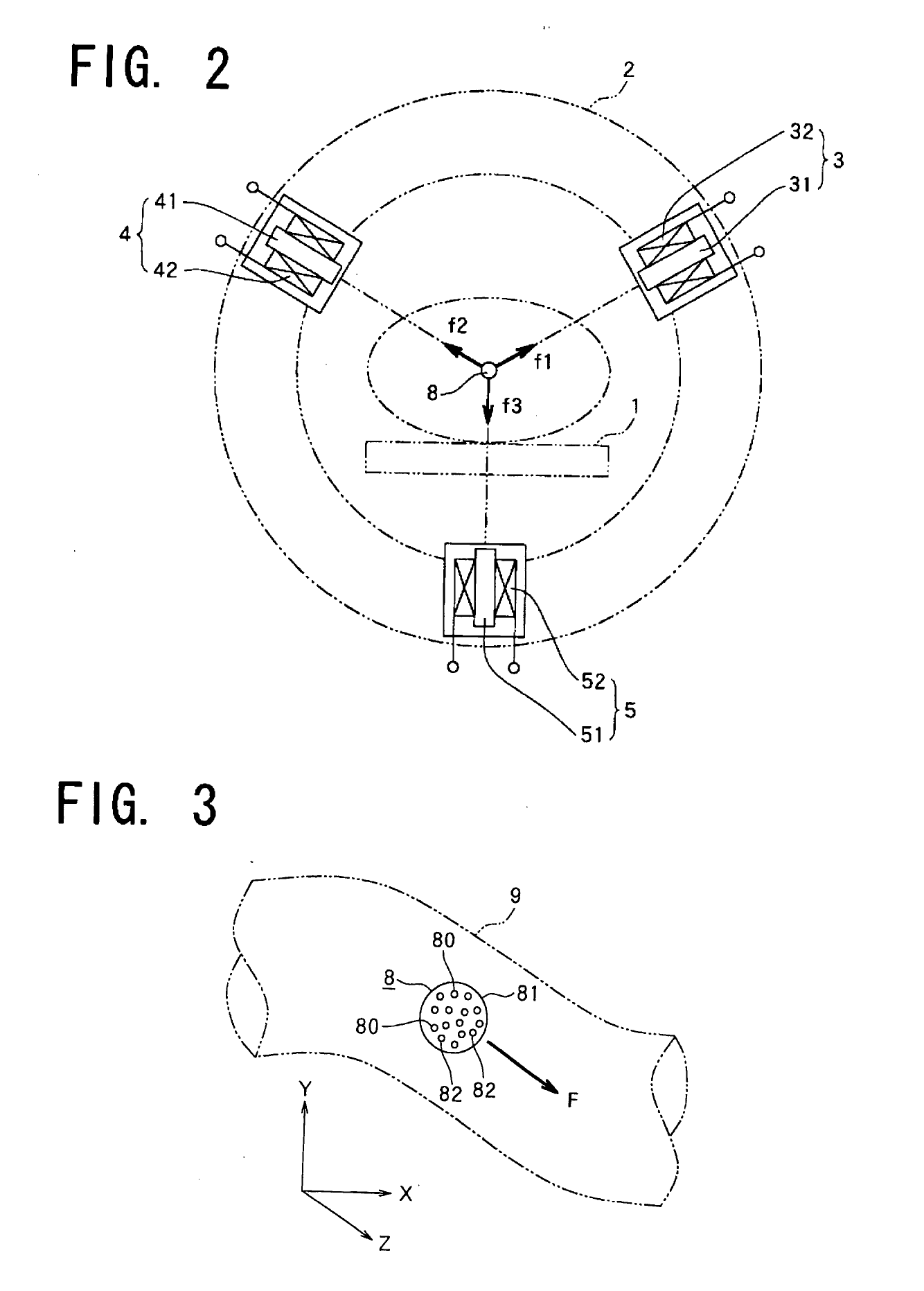

A three-dimensional guidance system of the present invention is for guiding a drug through a blood vessel of a patient to or close to a target affected part of an organ or the like, and for administering the drug to the affected part with a high local concentration. As shown in FIG. 3, a magnetic particle carrier 8, including magnetic particles 80 and drugs 82 enclosed in a microcapsule 81, is injected into a blood vessel 9 by an injector. Thereafter, a magnetic force F is applied to the magnetic particle carrier 8 to move the magnetic particle carrier 8 along the blood vessel 9.

The microcapsule 81 is formed to have an average diameter of less than 10 μm, with a hag-like body such as a liposome, for example, and is gradually absorbed in vivo over a period of about one month.

The magnetic particles 80 include a fine magnetic particle containing at least one element selected from iron, nickel, cobalt, manganese, arsenic, antimony, and bismuth, preferably including a fine particle of a magnetic iron oxide or magnetic ferrite, further preferably including a fine particle of a magnetic iron oxide.

A preferred magnetic iron oxide may be magnetite (Fe₃O₄), maghemite (γ-Fe₂O₃), or ferrous oxide (FeO). These magnets are each bioabsorbable, and will be gradually decomposed and metabolized in vivo. A preferred magnetic ferrite may be a magnetron-type ferrite such as barium ferrite (BaFe₆O₁₉), strontium ferrite (SrFe₆O₁₉), and lead ferrite (PbFe₆O₁₉).

The magnetic particles 80 desirably have an average diameter of about 10 nm to 9 μm, and thus can be enclosed in the microcapsule 81, exhibiting good magnetism.

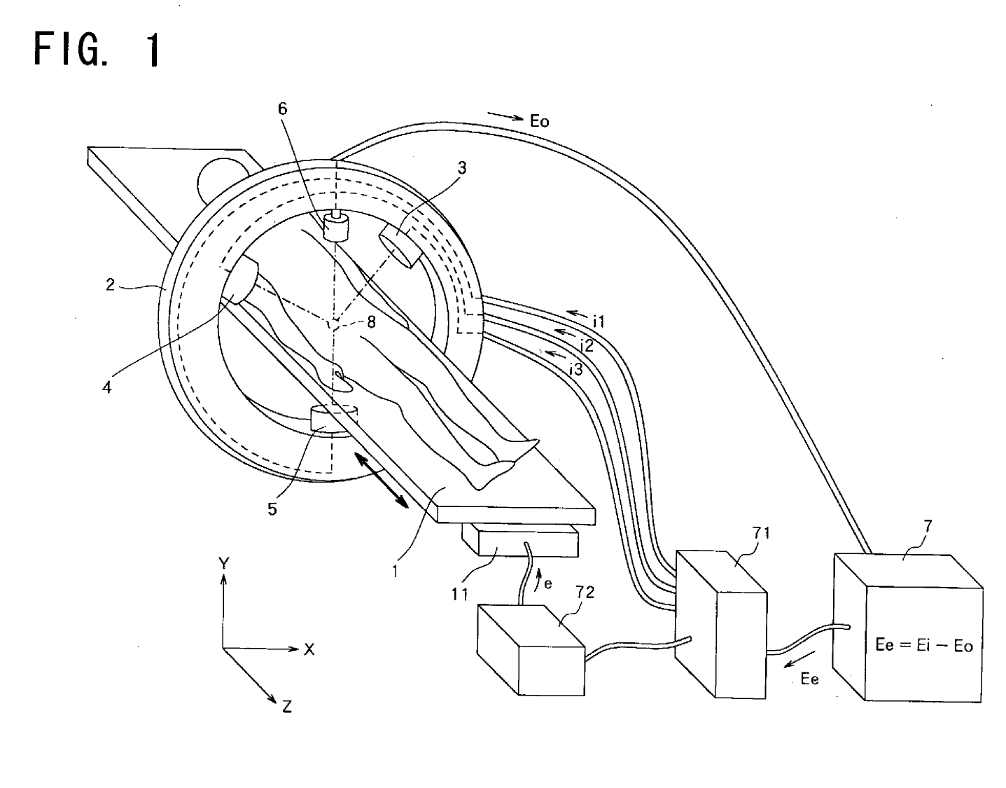

As shown in FIG. 1, the three-dimensional guidance system of the present invention includes a ring-shaped support 2, installed in a vertical plane including X-axis and Y-axis, and surrounding a bed 1 reciprocatingly driven in Z-axis direction by a bed drive motor 11. The ring-shaped support 2 has three electromagnets 3, 4, 5 regularly arranged for forming a magnetic field inside the ring-shaped support 2. The support 2 is not necessarily ring-shaped, but may have various shapes that allow the three electromagnets 3, 4, 5 to be supported.

As shown in FIG. 2, these three electromagnets 3, 4, 5 include cores 31, 41, 51 facing the center of the ring-shaped support 2, and coils 32, 42, 52 fitted around the cores, respectively. The coils 32, 42, 52 may be formed of not only general-purpose copper wires but also superconducting coils. The cores 31, 41, 51 may be omitted. Further, instead of the electromagnets 3, 4, 5, permanent magnets combined with electromagnets may be employed.

Energizing the three coils 32, 42, 52 allows the three electromagnets 3, 4, 5 to emit lines of magnetic force, and to have magnetic fields by the respective electromagnets superposed inside the ring-shaped support 2, forming a magnetic field with a flux density of about 0.01-10 T. The magnetic field varies in intensity and gradient depending on a current magnitude supplied to the three electromagnets 3, 4, 5. The internal magnetic particle carrier B experiences attractive forces f1, f2, f3 depending on the magnetic field intensity and gradient. As the bed 1 moves, the attractive forces f1, f2, f3 vary in direction to generate a Z-axis force on the magnetic particle carrier 8. Further, the magnetic particle carrier 8 experiences external forces such as gravity or fluid resistance due to the blood flow. The magnetic particle carrier 8 will experience a resultant force of these acting forces to move in the blood vessel.

Therefore, if a current to be supplied to the three electromagnets 3, 4, 5 and a voltage to be supplied to the bed drive motor 11 are controlled in accordance with an internal vascular route, the magnetic particle carrier 8 can be smoothly moved along the blood vessel 9 by the force F along the blood vessel 9 applied to the magnetic particle carrier 8, as shown in FIG. 3.

The three-dimensional guidance system of the present invention realizes the guidance of the magnetic particle carrier 8 along the blood vessel 9 by employing a feedback control as described later.

As shown in FIG. 1, the three electromagnets 3, 4, 5 are supplied with currents i1, i2, i3, respectively, from a current amplifier 71. The bed drive motor 11 is supplied with a drive voltage e from a motor power source 72. A controller 7 controls operations of the current amplifier 71 and motor power source 72.

The ring-shaped support 2 has a position detecting sensor 6 attached thereto for three-dimensionally detecting a position of the internal magnetic particle carrier 8. The position detecting sensor 6 includes a multichannel superconducting quantum interference device (SQUID), for example. The position detecting sensor 6 using a multichannel SQUID could determine a position of the magnetic particle carrier 8 from the in vivo magnetic field distribution with a millisecond time resolution and millimeter spatial resolution.

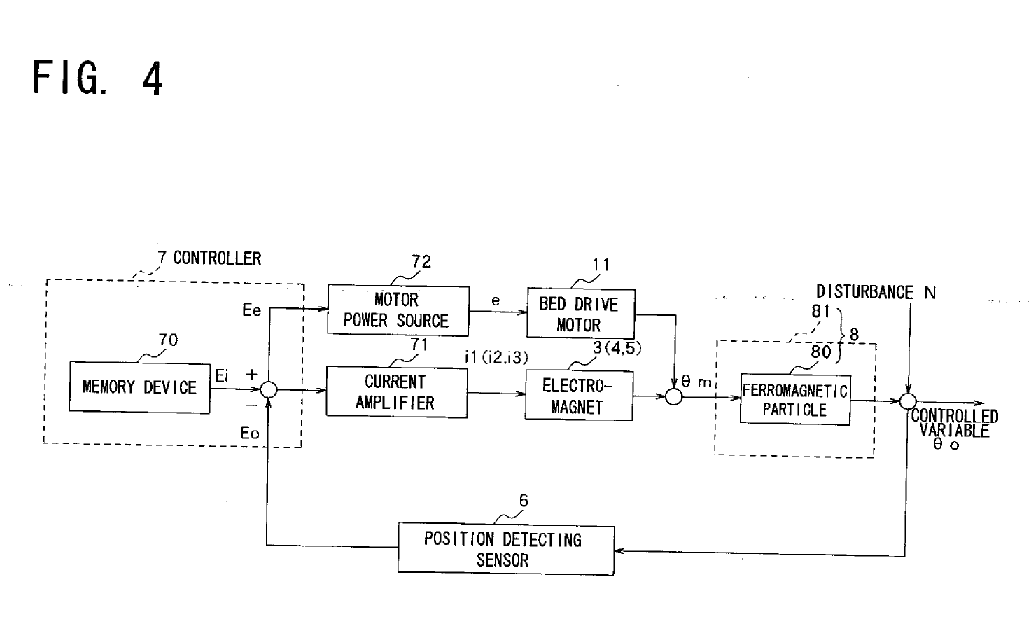

FIG. 4 illustrates a configuration of a control system in the above three-dimensional guidance system. The controller 7, including a computer, incorporates a memory device 70 such as a hard disk device. The memory device 70 stores a previously measured vascular route and target position of a patient as three-dimensional route data. The controller 7 derives a target value Ei for a position of the magnetic particle carrier 8 at a current time from the route data stored in the memory device 70. The controller 7 also calculates position data representing the current position of the magnetic particle carrier 8 from an output signal of the position detecting sensor 6. With the position data being a current value Eo, the controller 7 then calculates a deviation Ee (=Ei−Eo) of the current value Eo from the target value Ei, performs a PID control based on the deviation Ee to calculate currents i1, i2, i3 to be supplied to the three electromagnets 3, 4, 5, as well as a voltage e to be supplied to the bed drive motor 11, further, in accordance with the calculation result, prepares a control signal to be supplied to the current amplifier 71 and motor power source 72, and supplies the signal to the current amplifier 71 and motor power source 72.

Consequently, the three electromagnets 3, 4, 5 are supplied with the currents i1, i2, i3 from the current amplifier 71, while the bed drive motor 11 is supplied with the voltage e, resulting in an attractive force θm acting on the magnetic particle carrier 8. Besides gravity, the magnetic particle carrier 8 further experiences a disturbance N due to variation in blood flow, slight movement of the patient or the like. A resultant force of these acting forces moves the magnetic particle carrier 8, a position of which is detected by the position detecting sensor 6 as a controlled variable θo.

The above feedback control adjusts the currents i1, i2, i3 to be supplied to the three electromagnets 3, 4, 5 and the voltage e to be supplied to the bed drive motor 11 so as to bring the deviation Ee of the current value Eo from the target value Ei for the magnetic particle carrier 8, i.e., the position difference of the magnetic particle carrier 8 relative to the predetermined route along the blood vessel 9, close to zero. Consequently, the magnetic particle carrier 8 will move smoothly in the blood vessel 9 along the predetermined route.

According to Earnshaw's theorem, the magnetic particle carrier 8 cannot stand still stably at a constant position, but, because the present invention employs the feedback control as described above, the magnetic particle carrier 8 experiences a force along a predetermined route, so that the magnetic particle carrier 8 can be moved along the predetermined route.

The inventors conducted a computer simulation to guide magnetic particles to a heart left ventricle, in which they found, from a coordinate and velocity of the magnetic particles, a magnetic force and resistance force that act on the magnetic particles in a magnetic field and fluid field, and, by synthesizing these forces, calculated a trace of the magnetic particles in the fluid with the external magnetic field, confirming that this system can guide magnetic particles.

The three-dimensional guidance system of the present invention can guide the magnetic particle carrier 8 through the blood vessel 9 to a target organ or cell part without using a conventional tool such as a catheter, and can administer the drugs 82 included in the magnetic particle carrier 8 to the target organ or cell part with a high local concentration.

The above embodiment guides the magnetic particle carrier 8 using the three electromagnets 3, 4, 5, but, not only this, the guidance using the two electromagnets 3, 4 or four or more electromagnets is also possible. With respect to the guidance in Z-axis direction, not only moving the bed 1, but moving the three electromagnets 3, 4, 5 in Z-axis direction is also possible. Instead of controlling the currents of the electromagnets 3, 4, 5, controlling the movement of the bed 1 in X-axis and Y-axis directions is also possible.

The position detecting sensor 6 may be not only a multichannel SQUID but also a known position detecting sensor using a Hall element or the like. It may be effective to arrange a flux convergence member between each of the electromagnets and the patient, for a flux of lines of magnetic force generated from each of the electromagnets to converge at a local area.

The magnetic particles 80 of the magnetic particle carrier 8 to be guided in the blood vessel 9 may be formed of not only a magnetic metal but also a magnetic resin material. Not only the single magnetic particle carrier B is to be guided in the blood vessel 9, but the three-dimensional guidance system of the present invention is effective even when many magnetic particle carriers 8 move together.

Further, it is possible to employ a magnetic particle carrier 8 enclosing a biological molecule of protein, nucleic acid or the like, as well as the magnetic particles 80, in the microcapsule 81. Methods of carrying may include not only using a microcapsule but also attaching drugs 82 directly to magnetic particles 80, and attaching magnetic particles to vectors carrying drugs and genes.

Furthermore, the above three-dimensional guidance system can be embodied not only in a therapeutic unit for human bodies but also in various units for guiding an object along a channel in a structure.

Drug Delivery System

Described below is a drug delivery system for delivering drug particles, injected into an in vivo blood vessel, along the blood vessel close to an affected part. The drug particles are fine magnetic particles adhering to vectors, for example, and have a mean particle diameter of several tens nm to several μm, depending on an inner diameter of the blood vessel to be passed through.

The drug delivery system of the present invention can be realized using the above three-dimensional guidance system, for example, in which the magnetic field forming device includes a super-conducting magnet in order to form a satisfactory magnetic field gradient (for example, 70 T/m). The system controls the intensity and gradient of a magnetic field formed inside the human body, and guides the drug particles along a predetermined vascular route close to an affected part, where the particles can accumulate and aggregate.

For example, FIG. 5 shows a blood vessel branching from one main vessel B0 into two branch vessels B1, B2, where a magnetic field gradient where the magnetic field increases in strength from the inside of the blood vessel toward the outside is formed near one branch vessel B2 into which drug particles P are to be sent, whereby the drug particles concentratedly flow into the one branch vessel B2.

The inventors made an experimental system simulating the blood vessel shown in FIG. 5, and conducted an experiment to form the magnetic field gradient by arranging a permanent magnet M at a base end of the branch vessel B2 to observe a flow of the particles P. The experiment employed a tube with an inner diameter of 3 mm, and a fluid (H₂O) with a flow velocity of 10 cm/s, with three kinds of ferromagnetic particles (γ-Fe₂O₃) with each mean particle diameter of 44 μm, 2 μm, and 30 nm, and a columnar magnet M with a surface flux density of 0.1 T and an outer diameter of 4 mm. The result of the experiment confirmed that the particles P of every particle diameter flowed from the main vessel B0 into the intended branch vessel B2, regardless of an intratubular flow F, as shown in FIG. 5.

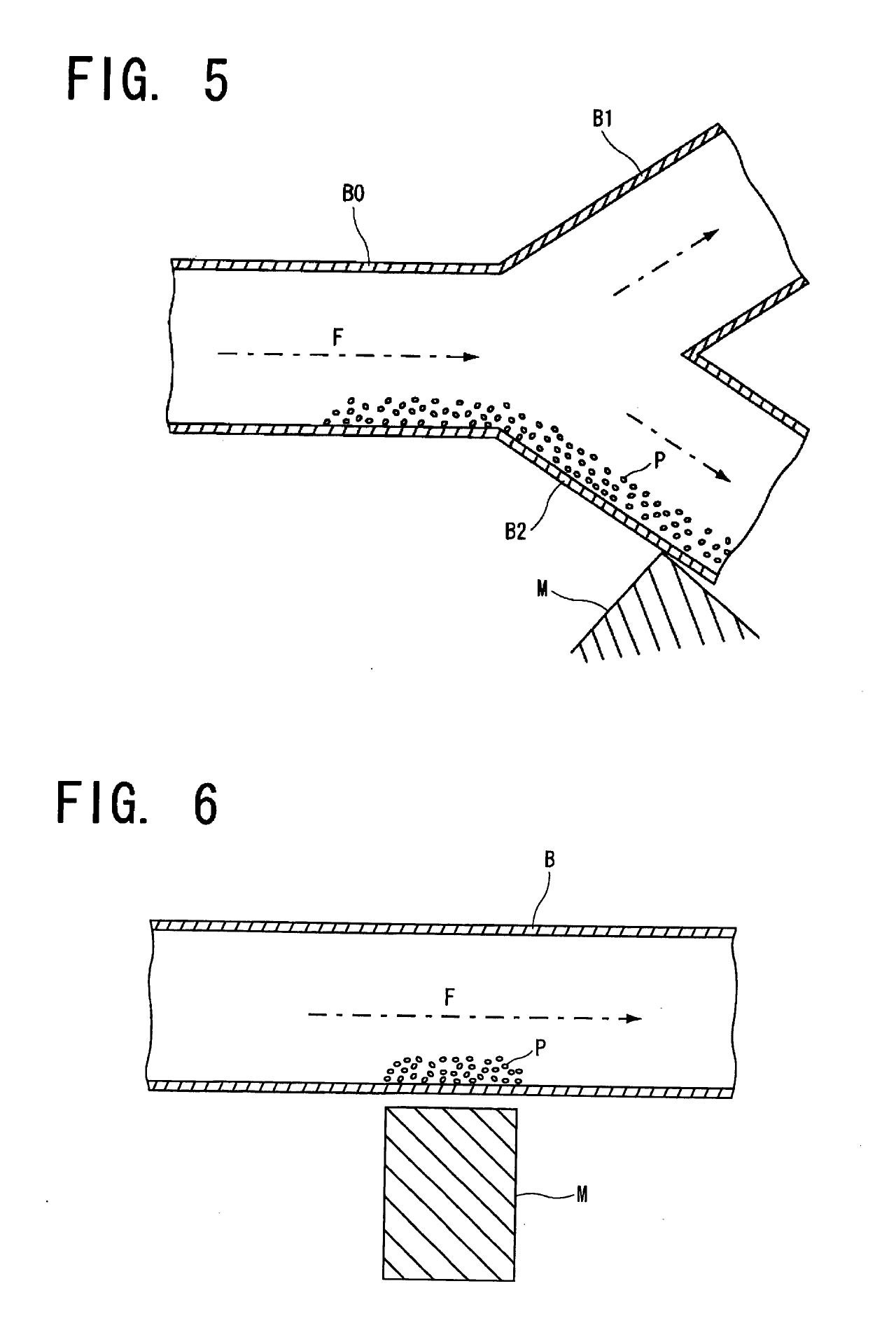

When, as shown in FIG. 6, drug particles accumulate on an inner wall of a blood vessel B near an affected part, a magnetic field gradient is formed where the magnetic field increases in strength from the inside of the blood vessel toward the outside. This allows the intravascular drug particles to aggregate near the affected part, and the drug to be administered to the affected part with a high local concentration.

The inventors made an experimental system simulating the blood vessel shown in FIG. 6, and conducted an experiment to form the magnetic field gradient by arranging a permanent magnet M to face an outer wall of the blood vessel B to observe a flow of the particles P. The experiment employed a tube with an inner diameter of 3 mm, and a fluid (H₂O) with a flow velocity of 10 cm/s, with three kinds of ferromagnetic particles (γ-Fe₂O₃) with each mean particle diameter of 44 μm, 2 μm, and 30 nm, and a columnar magnet M with a surface flux density of 0.1 T and an outer diameter of 4 mm. The result of the experiment confirmed that the particles P of every particle diameter accumulated and aggregated at an opposed position to the magnet M, regardless of an intratubular flow F, as shown in FIG. 6.

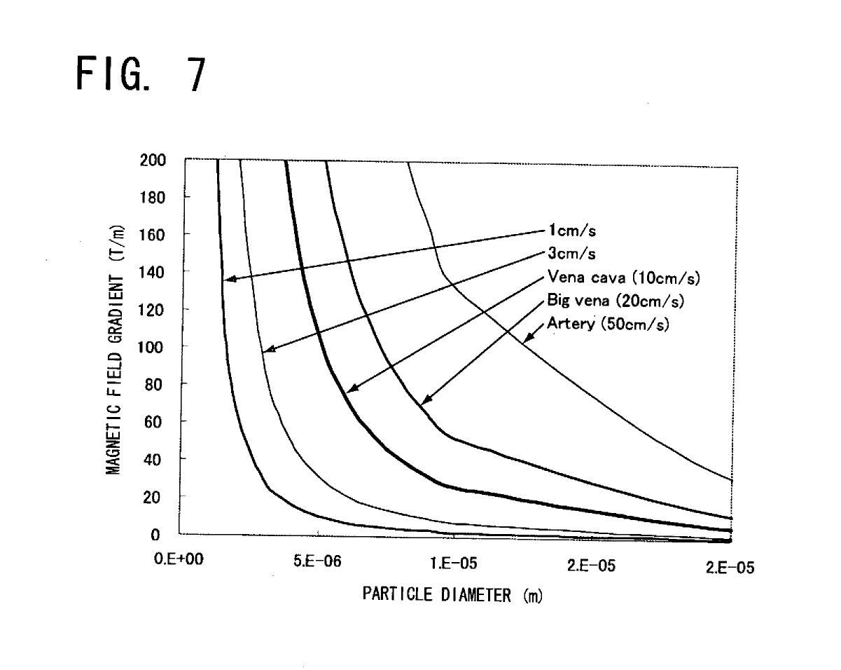

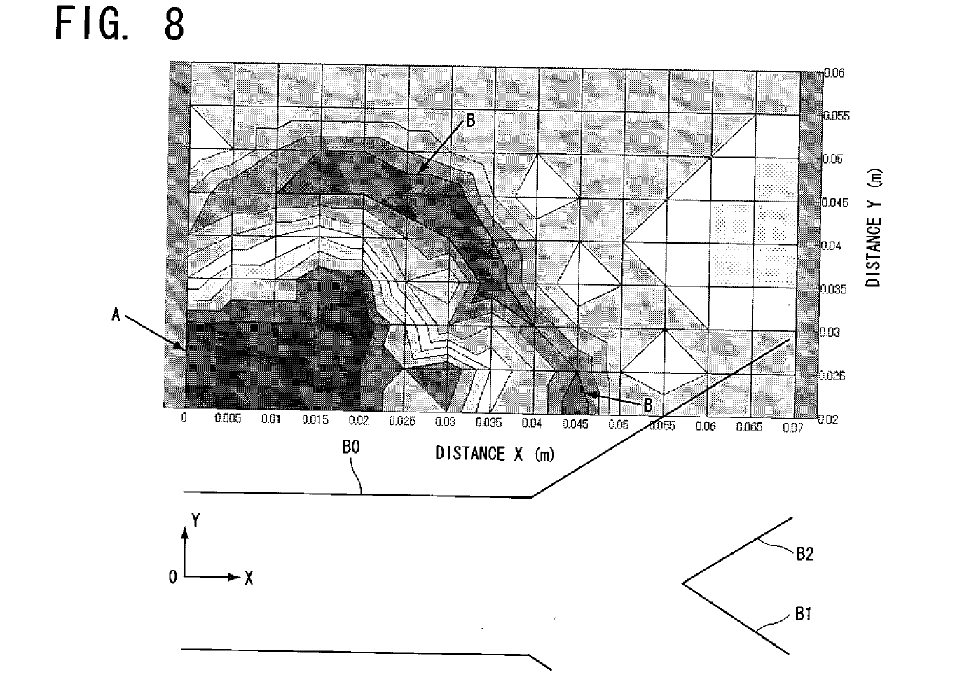

FIG. 7 shows a result of analyzing relationships between the drug particle diameter and magnetic field gradient, necessary for the drug particles to accumulate at a certain intravascular position as shown in FIG. 6. The analysis required that the magnetic force acting on the intravascular magnetic particles balance with the drag force acting on the magnetic particles due to the blood flow, and provided the relationships between the particle diameter and magnetic field gradient, with the flow velocity as a parameter. As apparent from FIG. 7, it is understood, for example, that drug particles with a diameter of 5 μm need a magnetic field gradient of 80-100 T/m in order to accumulate at a certain position in the vena cava where the blood flow velocity is 10 cm/s. However, the flow velocity significantly lowers near the vascular inner wall, and lowers a necessary magnetic field gradient accordingly. For example, if the flow velocity lowers to 3 cm/s, the drug particles can accumulate with a smaller magnetic field gradient of 40 T/m or less, which is well feasible with a super-conducting magnet.

FIG. 8 shows a result of a computer simulation to trace magnetic particles flows through a blood vessel branching from a main vessel B0 into two branch vessels B1, B2, and to determine a magnetic field position necessary for the particles to selectively flow into one branch vessel B2. The simulation built a finite element model of the blood vessel, where nine magnetic particles (diameter 2 μm) were arranged at intervals of 0.2 cm on the same X-coordinate of the main vessel B0, and these nine magnetic particles were traced. Then examined was a relationship between the relative position vector of the magnetic field to the fluid field (i.e., the magnetic field position) and the magnetic particles trace result.

As a result, when the magnetic field was placed in an area A shown in FIG. 8, all the nine particles accumulated in the main vessel B0, without flowing into any branch vessel. In contrast, when the magnetic field was placed in an area B shown in FIG. 8, all the nine particles flowed into the intended branch vessel B2. Thus, the magnetic particles accumulate at a certain position, or selectively flow into one of the branch vessels, depending on the magnetic field position. This result indicates that adjusting the magnetic field forming device position relative to the blood vessel allows the magnetic particles to accumulate at a certain position, or to selectively flow into an intended branch vessel. The adjustment of the magnetic field forming device position relative to the blood vessel can be realized, for example, by the three-dimensional guidance system of the present invention shown in FIG. 1.

As described above, the drug delivery system of the present invention allows drug particles, injected into a vein, for example, by an injector or the like, to selectively pass through one of the branches of a vascular system including veins and arteries, and to be delivered along a predetermined vascular route to or close to an affected part, where the intravascular drug particles can accumulate and aggregate. This allows the drug to be administered to the affected part with a high local concentration.

1. A three-dimensional guidance system for guiding a magnetic particle carrier along a channel extending in a certain route in three-dimensional space, comprising a magnetic field forming device for forming a magnetic field in the space having the channel therein, and a controller for controlling the magnetic field forming device operation, wherein the three-dimensional guidance system guides the magnetic particle carrier along the channel by controlling the magnetic field intensity and gradient of the magnetic field formed by the magnetic field forming device.

2. A three-dimensional guidance system for guiding a magnetic particle carrier along a channel extending in a certain route in three-dimensional space, comprising a magnetic field forming device for forming a magnetic field in the space having the channel therein, a controller for controlling the magnetic field forming device operation, and a position detecting sensor for detecting the magnetic particle carrier position in the channel, wherein the three-dimensional guidance system guides the magnetic particle carrier along the channel by feedback-controlling the magnetic field intensity and gradient of the magnetic field formed by the magnetic field forming device based on the magnetic particle carrier position detected by the position detecting sensor.

3. A three-dimensional guidance system for guiding a magnetic particle carrier along a channel extending in a certain route in three-dimensional space, comprising a position detecting sensor for detecting the magnetic particle carrier position in the channel, a plurality of electromagnets arranged to surround the channel, a driver for displacing the plurality of electromagnets relative to the channel in a direction penetrating a plane having the electromagnets arranged therein, and a control circuit for controlling a current to be supplied to the plurality of electromagnets and a drive signal to be supplied to the driver, the control circuit comprising:
data holding means for holding the channel route as three-dimensional route data; and
feedback control means for feedback-controlling the current to be supplied to the plurality of electromagnets and the drive signal to be supplied to the driver, based on a deviation of position data representing the current position of the magnetic particle carrier detected by the position detecting sensor from the route data held by the data holding means.

4. A three-dimensional guidance system for guiding a magnetic particle carrier, injected into a blood vessel in a body, along the blood vessel, comprising a magnetic field forming device for forming a magnetic field in space having the body therein, and a controller for controlling the magnetic field forming device operation, wherein the three-dimensional guidance system guides the magnetic particle carrier along the blood vessel by controlling the magnetic field intensity and gradient of the magnetic field formed by the magnetic field forming device.

5. A three-dimensional guidance system for guiding a magnetic particle carrier, injected into a blood vessel in a body, along the blood vessel, comprising a magnetic field forming device for forming a magnetic field in space having the body therein, a controller for controlling the magnetic field forming device operation, and a position detecting sensor for detecting the magnetic particle carrier position in the blood vessel, wherein the three-dimensional guidance system guides the magnetic particle carrier along the blood vessel by feedback-controlling the magnetic field intensity and gradient of the magnetic field formed by the magnetic field forming device, based on the magnetic particle carrier position detected by the position detecting sensor.

6. A three-dimensional guidance system for guiding a magnetic particle carrier, injected into a blood vessel in a body, along the blood vessel, comprising a position detecting sensor for detecting the magnetic particle carrier position in the blood vessel, a plurality of electromagnets to be arranged to surround the body, a driver for displacing the plurality of electromagnets relative to the body in a direction penetrating a plane having the plurality of electromagnets arranged therein, and a control circuit for controlling a current to be supplied to the plurality of electromagnets and a drive signal to be supplied to the driver, the control circuit comprising:
data holding means for holding the blood vessel route extending in the body as three-dimensional route data; and
feedback control means for feedback-controlling the current to be supplied to the plurality of electromagnets and the drive signal to be supplied to the driver, based on a deviation of position data representing the current position of the magnetic particle carrier detected by the position detecting sensor from the route data held by the data holding means.

7. The tree-dimensional guidance system according to claim 6, wherein the driver is for moving a bed one-dimensionally with a bed drive motor, and the plurality of electromagnets are arranged to surround the bed in a plane perpendicular to the bed moving direction.

8. The three-dimensional guidance system according to claim 7, wherein the feedback control means of the control circuit prepares a current signal depending on the current to be supplied to the plurality of electromagnets, and a voltage signal depending on the drive signal to be supplied to the driver, based on the deviation, and supplies the current signal through a current amplifier to each of the electromagnets, and the voltage signal to the bed drive motor.

9. The three-dimensional guidance system according to any of claims 4 to 8, wherein the magnetic particle carrier comprises a drug or biological molecule carrying a magnetic particle.

10. The three-dimensional guidance system according to any of claims 4 to 8, wherein the magnetic particle carrier comprises a microcapsule enclosing a drug or biological molecule with a magnetic particle.

11. The three-dimensional guidance system according to any of claims 4 to 8, wherein the magnetic particle contains one or more metals selected from iron, nickel and cobalt, or compounds of these metals.

12. A three-dimensional guidance method for guiding a magnetic particle carrier along a channel extending in a certain route in three-dimensional space, comprising forming a magnetic field in the space having the channel therein, controlling the magnetic field intensity and gradient of the magnetic field, and thereby guiding the magnetic particle carrier along the channel.

13. A drug delivery system for delivering drug particles, injected into a blood vessel in a body, along the blood vessel close to an affected part, each drug particle comprising a drug or biological molecule carrying a magnetic particle, the drug delivery system comprising a magnetic field forming device for forming a magnetic field in space having the body therein, and a controller for controlling the magnetic field forming device operation, wherein the system controls the magnetic field intensity and gradient of the magnetic field formed by the magnetic field forming device, and thereby guides the drug particles along a predetermined vascular route close to the affected part, where the particles accumulate and aggregate.

14. The drug delivery system according to claim 13, wherein the controller sets the magnetic field gradient magnitude with the drug particle diameter and intravascular flow velocity being parameters.

15. The drug delivery system according to claim 13 or 14, wherein the magnetic field forming device comprises a super-conducting magnet.

16. The drug delivery system according to claim 12 or 14, wherein a driver is provided for varying the magnetic field forming device position relative to the body.

17. A drug delivery method for delivering drug particles, injected into a blood vessel in a body, along the blood vessel close to an affected pan, each drug particle comprising a drug or biological molecule carrying a magnetic particle, the method comprising forming a magnetic field in space having the blood vessel therein, controlling the magnetic field intensity and gradient of the magnetic field, and thereby guiding the drug particles along a predetermined vascular route close to the affected part, where the particles accumulate and aggregate.

18. The drug delivery method according to claim 17, wherein a magnetic field gradient where the magnetic field increases in strength from the inside of the blood vessel toward the outside is formed near the affected part in the body, whereby the drugs in the blood vessel accumulate and aggregate near the affected part.

19. The drug delivery method according to claim 17, wherein, at a vascular furcation from one main vessel to a plurality of branch vessels, a magnetic field gradient where the magnetic field increases in strength from the inside of the blood vessel toward the outside is formed near one branch vessel into which the drug particles are to be sent, whereby the drug particles concentratedly flow into the one branch vessel.

20. The drug delivery method according to any of claims 17 to 19, wherein the magnetic field gradient magnitude is set with the drug particle diameter and intravascular flow velocity being parameters.

21. A guidance system for guiding a magnetic particle carrier along a channel extending in a certain route, the channel having a fluid flowing therethrough and having a furcation from one main tube to a plurality of branch tubes, the system comprising a magnet arranged in space on the upstream side to the furcation and with a larger distance from the main tube with a further distance from the furcation, and controlling the intensity and gradient of a magnetic field formed by the magnet to thereby guide the magnetic particle carrier from the main tube into one particular branch tube.

22. The guidance system according to claim 21, wherein the magnetic field gradient magnitude is set with the diameter of a magnetic particle constituting the magnetic particle carrier and the fluid velocity being parameters.

23. The guidance system according to claim 21 wherein the magnetic particle carrier is a drug particle comprising a drug or biological molecule carrying a magnetic particle.

24. The guidance system according to claim 23, wherein the magnetic particle carrier comprises a microcapsule enclosing the drug or biological molecule with the magnetic particle.

25. The guidance system according to claim 21, wherein the magnetic particle carrier comprises a magnetic particle containing one or more metals selected from iron, nickel and cobalt, or compounds of these metals.
